(19) United States
(12) Patent Application Publication
(10) Pub. No.: US 2024/0079087 A1
(43) Pub. Date: Mar. 7, 2024

(54) IMPLEMENTATION METHOD FOR ULTRASENSITIVE BRINK CONTROL FOR DELAYED ENZYMATIC REACTION BASED ON DNA STRAND DISPLACEMENT

(71) Applicant: DALIAN UNIVERSITY, Dalian (CN)

(72) Inventors: Qiang ZHANG, Dalian (CN); Hui LV, Dalian (CN); Shihua ZHOU, Dalian (CN); Bin WANG, Dalian (CN); Yijun XIAO, Dalian (CN); XingAn WANG, Dalian (CN)

(73) Assignee: DALIAN UNIVERSITY, Dalian (CN)

(21) Appl. No.: 18/457,348

(22) Filed: Aug. 29, 2023

Related U.S. Application Data
(63) Continuation of application No. PCT/CN2023/101281, filed on Jun. 20, 2023.

(30) Foreign Application Priority Data
Aug. 25, 2022 (CN) 202211026723X

(51) Int. Cl. G16B 20/10; G05B 13/04; G16B 5/00

(52) U.S. Cl. CPC G16B 20/10; G05B 13/042; G16B 5/00

(57) ABSTRACT

Disclosed is an implementation method for ultrasensitive Brink control for a delayed enzymatic reaction based on DNA strand displacement (DSD), relating to the field of feedback control technology based on DSD in biological systems. The method includes obtaining an enzymatic reaction process model with time delay; constructing a CRN-based Brink controller; obtaining a static mapping expression between an output of the Brink controller and an output of the system under a steady state condition; constructing a Brink controller by DSD reaction; obtaining a time delay representation, and applying it to DNA implementations of the enzymatic reaction process model; and combined with the Brink controller, controlling a delayed enzymatic reaction process model. The present invention is structurally free of subtraction, reduces the number of abstract chemical reactions required to implement, and greatly simplifies DNA implementation.

### CROSS-REFERENCE TO RELATED APPLICATIONS

This application claims the priority of the Chinese patent application submitted to the Chinese Patent Office on Aug. 25, 2022, with application number 202211026723. X and invention title “Implementation method for ultrasensitive Brink control for a delayed enzymatic reaction based on DNA strand displacement”, all of which are incorporated herein by reference.

### TECHNICAL FIELD

The present invention relates to the field of feedback control technology based on DNA strand displacement in biological systems, in particular to an implementation method for ultrasensitive Brink control for a delayed enzymatic reaction based on DNA strand displacement.

### BACKGROUND

Chemical reaction networks (CRNs) are commonly used to represent feedback control systems to reflect the performance of biomolecular feedback control circuits. Meanwhile, DNA molecules are widely regarded as the ideal engineering materials for constructing molecular devices based on CRNs. In particular, the DNA strand displacement (DSD) reaction has become a means for formal programming and analyzing DNA devices. Therefore, in designing applications such as biochemical controllers, constructing CRNs to represent the kinetics of the system becomes a major goal. Combined with the DSD mechanism, digital circuits, signal processing calculations, and simulation can be realized. The existing CRN-based controllers mostly use the dual-rail representation method, which directly leads to a large increase in the number of CRNs required for the controller implementation, thus increasing the complexity of DNA implementation.

The CRN provides an abstract representation of complex biochemical processes, which provides an important premise for constructing a CRN representation of each module in the control system and reflecting the performance of the biomolecular feedback control circuit. Samaniego, C. C., and Franco, E., published an article in Cell Systems, 12 (3), 272-288 in 2021 titled “Ultrasensitive molecular controllers for quasi-integral feedback”, which utilized a modular design strategy to construct a molecular controller, the Brink controller, and applied it to regulate the expression of target RNA or proteins. The modular strategy involved in the construction of Brink controllers is based on two design principles: the use of ultrasensitive response and responded tunable threshold. In addition, Fern, J, Scalise, D, Cangialosi, A, Howie, D, Potters, L, and Schulman, R. published an article in ACS synthetic biology, 6 (2), 190-193 in 2017 titled “DNA strand-displacement time circuits”, which constructed a timer circuit using abstract chemical reactions that can coordinate different in vitro chemical events without external stimuli, such as for pre specifying specific species for a delayed release.

The existing CRN-based controllers have undergone a series of evolutions, such as PI controller, PID controller, and nonlinear QSM controller, and have also completed the corresponding DNA implementation. However, these controllers are structured such that there is a subtraction between the signals so that the design of CRNs depends on the dual-rail representation method, which increases the number of CRNs required for the implemention and increases the difficulty of DNA implementation.

### SUMMARY

It is an object of the present invention to provide an implementation of an ultrasensitive biomolecular Brink controller based on DNA strand displacement that achieves an ultrasensitive input-output response with as few chemical reactions as possible.

To achieve the above objectives, this application proposes an implementation method for delayed enzymatic reaction ultrasensitive Brink control based on DNA strand displacement, including:

describing an enzymatic reaction process using single-molecule and bimolecular chemical reactions, and introducing a time delay factor to obtain an enzymatic reaction process model with time delay;

constructing a CRN-based Brink controller;

obtaining a static mapping expression between an output of the Brink controller and an output of the system under a steady state condition so as to obtain an analytical condition ensuring the performance of the controller;

constructing a Brink controller by DNA strand displacement reaction; and

obtaining a time delay representation through DNA strand displacement mechanism based on a delayed substance and a compensation mechanism, and applying the time delay representation to a DNA implementation of the enzymatic reaction process model; at the same time, combined with the constructed Brink controller, controlling a delayed enzymatic reaction process model.

Further, describing an enzymatic reaction process using single-molecule and bimolecular chemical reactions, specifically includes:

$$S + B \begin{matrix}{⟶}^{{K}_{1}} \\ {⟵}_{{K}_{2}}\end{matrix} X$$

$$X {⟶}^{{k}_{3}} P + B$$

$$P {⟶}^{{k}_{4}} ∅$$

where S and B represent a substrate and an enzyme, respectively, X and P represent an enzyme-substrate complex and an output substance, respectively.

k₁, k₂, k₃, k₄ are all reaction rates, and Ø represents a degradation reaction.

Further, constructing the enzymatic reaction process model with time delay specifically includes:

$$S + B {⟶}^{{k}_{1}} X$$

$$X {⟶}^{{k}_{2}} S + B$$

$$X {⟶}^{{k}_{3}} P + B$$

$$P {⟶}^{{k}_{4} , τ} ∅$$

where parameter τ represents a cumulative time delay present in the production of the output substance P.

Further, the CRN-based controller is represented as:

$$R {⟶}^{{k}_{c}} R + {R}_{r}$$

$$Y {⟶}^{{θ}_{c}} Y + {R}_{y}$$

$${R}_{r} + {R}_{y} {⟶}^{{γ}_{c}} {R}_{r} \cdot {R}_{y}$$

$${R}_{r} {⟶}^{{ϕ}_{c}} ∅$$

$${R}_{y} {⟶}^{{ϕ}_{c}} ∅$$

$${R}_{r} + {U}^{*} {⟶}^{{α}_{c}} U + {R}_{r} \cdot {R}_{y}$$

$${R}_{y} + U {⟶}^{{β}_{c}} {U}^{*}$$

where parameters R and Y are inputs to the Brink controller and U represents an output; parameters k_{c}, θ_{c} and α_{c} represent the catalysis rate; γ_{c} and β_{c} represents the binding rate; ϕ_{c} represents the degradation rate; further, the parameter R produces substance Rᵣ, which in turn is reacted with U* to form U; parameter Y produces substance R_{y}, which in turn is reacted with U to form U*; at the same time, signals Rᵣ and R_{y} are bound to form a complex Rᵣ·R_{y} that does not interact with any other substance, i.e. there is an inverse functional mechanism between the two different input parameters R and Y of the Brink controller; the Brink controller uses the signals Rᵣ and R_{y} as activator and deactivator, respectively;

Combined with the mass action kinetics (MAKs), the corresponding ODEs equations are:

$$\frac{{d [ {R}_{y} ]}_{t}}{dt} = {{k}_{c} [ R ]}_{t} - {{{γ}_{c} [ {R}_{r} ]}_{t} [ {R}_{y} ]}_{t} - {{ϕ}_{c} [ {R}_{r} ]}_{t} - {{{α}_{c} [ {R}_{r} ]}_{t} [ {U}^{*} ]}_{t}$$

$$\frac{{d [ {R}_{y} ]}_{t}}{dt} = {{θ}_{c} [ Y ]}_{t} - {{{γ}_{c} [ {R}_{r} ]}_{t} [ {R}_{y} ]}_{t} - {{ϕ}_{c} [ {R}_{y} ]}_{t} - {{{β}_{c} [ {R}_{y} ]}_{t} [ U ]}_{t}$$

$$\frac{{d [ {U}^{*} ]}_{t}}{dt} = - {{{α}_{c} [ {R}_{r} ]}_{t} [ {U}^{*} ]}_{t} + {{{β}_{c} [ {R}_{y} ]}_{t} [ U ]}_{t}$$

$$\frac{{d [ U ]}_{t}}{dt} = {{{α}_{c} [ {R}_{r} ]}_{t} [ {U}^{*} ]}_{t} - {{{β}_{c} [ {R}_{y} ]}_{t} [ U ]}_{t}$$

(d[U*]ₜ/dt)+(d[U]ₜ/dt)=0 obtained from the differential equation indicates that the total mass U+U* is conserved during the process of time evolution.

Further, obtaining a static mapping expression between an output of the Brink controller and an output of the system under a steady state condition specifically includes:

assuming the Brink controller has achieved steady-state output, the following results are obtained:

k_{c}[R]ₜ−γ_{c}[Rᵣ]ₜ[R_{y}]ₜ−ϕ_{c}[Rᵣ]ₜ−α_{c}[Rᵣ]ₜ[U*]ₜ=0

θ_{c}[Y]ₜ−γ_{c}[Rᵣ]ₜ[R_{y}]ₜ−ϕ_{c}[R_{y}]ₜ−β_{c}[R_{y}]ₜ[U]ₜ=0

−α_{c}[Rᵣ]ₜ[U*]ₜ+β_{c}[R_{y}]ₜ[U]ₜ=0

assuming that the Brink controller reference input R is constant, the following constraints are obtained:

$$[ \overline{Y} ] = \frac{{k}_{c} [ R ] - {α}_{c} [ {\overline{R}}_{r} ] [ {\overline{U}}^{*} ] + {β}_{c} [ {\overline{R}}_{y} ] [ \overline{U} ] - {ϕ}_{c} [ {\overline{R}}_{r} ] + {ϕ}_{c} [ {\overline{R}}_{y} ]}{{θ}_{c}} = \frac{{k}_{c}}{{θ}_{c}} [ R ] + \frac{{ϕ}_{c}}{{θ}_{c}} ( [ {\overline{R}}_{y} ] - [ {\overline{R}}_{r} ] )$$

where the signal [⋅̅] is indicative of the concentration of the substance ⋅ at a steady state.

Further, constructing the Brink controller by using the DNA strand displacement reaction, specifically includes: set i, x, y, and z as variables, where i∈(1, 2, . . . , 12), x∈(1, 2, . . . , 8), y∈(1, 2, 3, 4), z∈(1, 2, . . . , 9);

for reactions

$$R {⟶}^{{k}_{c}} R + {R}_{r} and Y {⟶}^{{θ}_{c}} Y + {R}_{y} ,$$

there is a common DSD implementation mechanism between the two; these two reactions are converted into:

$$\begin{matrix}R + {G}_{1} {⟶}^{{q}_{1}} ∅ + {O}_{1} \\ {O}_{1} + {T}_{1} {⟶}^{{q}_{max}} R + {R}_{r}\end{matrix} \} , {q}_{2} = \frac{{K}_{c}}{{C}_{max}}$$

$$\begin{matrix}Y + {G}_{2} {⟶}^{{q}_{2}} ∅ + {O}_{2} \\ {O}_{2} + {T}_{2} {⟶}^{{q}_{max}} Y + {R}_{y}\end{matrix} \} , {q}_{2} = \frac{{θ}_{c}}{{C}_{max}}$$

At the same time, there is also an identical realization mechanism between the reactions

$${R}_{r} {⟶}^{{ϕ}_{c}} ∅ and {R}_{y} {⟶}^{{ϕ}_{c}} ∅ ,$$

and the transformation is represented as:

$$\begin{matrix}{R}_{r} + {G}_{3} {⟶}^{{q}_{3}} ∅ \\ {R}_{y} + {G}_{4} {⟶}^{{q}_{4}} ∅\end{matrix} \} , {q}_{3} = {q}_{4} = \frac{{ϕ}_{c}}{{C}_{max}}$$

for the reactions

$${R}_{r} + {R}_{y} {\to }^{{γ}_{c}} {R}_{r} \cdot {R}_{y} , {R}_{r} + {U}^{*} {\to }^{{α}_{c}} U + {R}_{r} \cdot {R}_{y} and {R}_{y} + U {\to }^{{β}_{c}} {U}^{*} ,$$

the corresponding DNA implementations are represented as:

$$\begin{matrix}{R}_{r} + {L}_{1} {⇄}_{{q}_{max}}^{{q}_{5}} {H}_{1} + {B}_{1} \\ {R}_{y} + {H}_{1} {\to }^{{q}_{max}} {O}_{3} + ∅ \\ {O}_{3} + {T}_{3} {\to }^{{q}_{max}} {R}_{r} \cdot {R}_{y} + ∅\end{matrix} \} , {q}_{5} = {γ}_{c}$$

$$\begin{matrix}{R}_{r} + {L}_{2} {⇄}_{{q}_{max}}^{{q}_{6}} {H}_{2} + {B}_{2} \\ {U}^{*} + {H}_{2} {\to }^{{q}_{max}} {O}_{4} + ∅ \\ {O}_{3} + {T}_{4} {\to }^{{q}_{max}} {R}_{r} \cdot {R}_{y} + U\end{matrix} \} , {q}_{6} = {α}_{c}$$

$$\begin{matrix}{R}_{y} + {L}_{3} {⇄}_{{q}_{max}}^{{q}_{7}} {H}_{3} + {B}_{3} \\ {U}^{*} + {H}_{2} {\to }^{{q}_{max}} {O}_{5} + ∅ \\ {O}_{5} + {T}_{5} {\to }^{{q}_{max}} {U}^{*} + ∅\end{matrix} \} , {q}_{7} = {β}_{c}$$

where Gₓ, Tₓ and L_{y} represent auxiliary substances involved in the reaction; O_{z} and H_{y} represent intermediate products; B_{y} represents inert wastes produced by the reaction which do not interact with other substances; further, Cₘₐₓ represents the initial concentration of the auxiliary substance; qₘₐₓ represents the reaction rate of maximum strand displacement; qᵢ represents the reaction rate of the corresponding DNA implementation.

Further, obtaining the time delay representation through the DNA strand displacement mechanism based on the delayed substance and a compensation mechanism specifically includes:

the time delay is represented by a circuit consisting of two abstract chemical reactions taking place simultaneously, the implementation of which is based on the participation of a delaying substance D, and described by the following reactions:

$${∅}_{source} {\to }^{{k}_{prod}} O$$

$$O + D {\to }^{{k}_{delay}} {∅}_{waste}$$

where parameters k_{prod} and k_{delay} are rate constants; in a first stage, substance O is produced at a constant rate; in the second stage, when the substance O is bound to the delayed substance D, it is rapidly converted into waste Ø_{waste}; the time taken for the substance O to consume substance D is taken as the delay time, and the delay effect thereof depends on the initial concentration of the delay substance D.

Further, by applying the time delay representation to the DNA implementation of the enzymatic reaction process model, the enzymatic reaction model is rewritten as:

$$S + B {\to }^{{k}_{1}} X$$

$$X {\to }^{{k}_{2}} S + B$$

$$X {\to }^{{k}_{3}} P + B$$

$$P + {D}_{1} {\to }^{{k}_{delay 1}} ∅$$

$$P {\to }^{{k}_{4}} ∅$$

where k_{delay1} represents a delayed reaction rate; combined with the mass action kinetics, the following results are obtained:

$$\frac{{d [ S ]}_{t}}{dt} = - {{{k}_{1} [ S ]}_{t} [ B ]}_{t} + {{k}_{2} [ X ]}_{t} + {[ U ]}_{t}$$

$$\frac{{d [ B ]}_{t}}{dt} = - {{{k}_{1} [ S ]}_{t} [ B ]}_{t} + {{k}_{2} [ X ]}_{t} + {{k}_{3} [ X ]}_{t}$$

$$\frac{{d [ X ]}_{t}}{dt} = {{{k}_{1} [ S ]}_{t} [ B ]}_{t} - {{k}_{2} [ X ]}_{t} - {{k}_{3} [ X ]}_{t}$$

$$\frac{{d [ P ]}_{t}}{dt} = {{k}_{3} [ X ]}_{t} - {{{k}_{delay 1} [ P ]}_{t} [ {D}_{1} ]}_{t} - {{k}_{4} [ P ]}_{t}$$

$$\frac{{d [ {D}_{1} ]}_{t}}{dt} = - {{{k}_{delay1} [ P ]}_{t} [ {D}_{1} ]}_{t}$$

Further, combined with the constructed Brink controller, controlling the delayed enzymatic reaction process model specifically includes:

converting reaction

$$X {\to }^{{k}_{3}} P + B$$

to:

$$\begin{matrix}X + {G}_{5} {\to }^{{q}_{8}} ∅ + {O}_{6} \\ {O}_{6} + {T}_{6} {\to }^{{q}_{max}} P + B\end{matrix} \} , {q}_{8} = \frac{{k}_{3}}{{C}_{max}}$$

converting degradation reaction

$$P {\to }^{{k}_{4}} ∅$$

to:

$$P + {G}_{6} {\to }^{{q}_{9}} ∅ , {q}_{9} = \frac{{k}_{4}}{{C}_{max}}$$

further, converting reaction

$$P + {D}_{1} {\to }^{{k}_{delay 1}} ∅$$

to:

$$\begin{matrix}P + {G}_{7} {\to }^{{q}_{10}} ∅ + {O}_{7} \\ {O}_{7} + {D}_{1} {\to }^{{q}_{max}} ∅\end{matrix} \} , {q}_{10} = \frac{{k}_{delay 1}}{{C}_{max}}$$

for reversible reaction

$$S + B {⇄}_{{K}_{2}}^{{K}_{1}} X ,$$

original reaction form is maintained when designing the DNA implementation.

Further, the enzymatic reaction process model of the Brink-based controller is adjusted by using the DSD mechanism; the proposed expression of DNA strand displacement with respect to time delay is improved, specifically as follows: the consumption of substance P in the enzymatic reaction is compensated by the following reaction mechanism to achieve the desired yield of output substance P:

$$P + F {\to }^{{k}_{pro 1}} 2 P$$

$$P {\to }^{{k}_{pro 2}} F$$

where kₚᵣₒ₁ and kₚᵣₒ₂ are both reaction rate constants and F is an additionally added reaction substance; combined with the mass action kinetics, the corresponding ordinary differential equations (ODEs) are obtained:

$$\frac{{d [ P ]}_{t}}{dt} = {{{k}_{pro 1} [ P ]}_{t} [ F ]}_{t} - {{k}_{pro 2} [ P ]}_{t}$$

$$\frac{{d [ F ]}_{t}}{dt} = - {{{k}_{pro 1} [ P ]}_{t} [ F ]}_{t} + {{k}_{pro 2} [ P ]}_{t}$$

Further, reaction

$$P + F {\to }^{{k}_{pro 1}} 2 P$$

is converted to:

$$\begin{matrix}P + {L}_{4} {⇄}_{{q}_{max}}^{{q}_{11}} {H}_{4} + {B}_{4} \\ F + {H}_{4} {\to }^{{q}_{max}} {O}_{8} + ∅ \\ {O}_{8} + {T}_{7} {\to }^{{q}_{max}} 2 P + ∅\end{matrix} \} , \qquad {q}_{11} = {k}_{p r o 1}$$

Reaction

$$P {\to }^{{k}_{p r o 2}} F$$

is converted to:

$$\begin{matrix}P + {G}_{8} {⟶}^{{q}_{12}} ∅ + {O}_{9} \\ {O}_{9} + {T}_{8} {\to }^{{q}_{max}} F + ∅\end{matrix} \} , {q}_{12} = \frac{{k}_{p r o 2}}{{C}_{max}}$$

The above technical solutions adopted by the present invention have the following advantages compared to existing technologies:

The Brink controller circumvents the limitation of the dual-rail representation method in CRNs design, structurally free of subtraction, reducing the number of abstract chemical reactions required to implement, and greatly simplifying the DNA implementation. Further, a delayed enzymatic reaction model based on CRNs is constructed by introducing a time delay factor. Considering the conversion representation between CRNs and DNA reactions, a representation scheme for a time delay in DNA strand displacement reactions is proposed. Finally, DNA strand displacement mechanism is used to control the enzymatic process under the Brink controller. Under the conditions of no delay and non-zero delay, the output substance of the enzymatic reaction process can approach the target level in a quasi-steady state.

### BRIEF DESCRIPTION OF DRAWINGS

FIG. 1 is an abstract representation of various chemical substances involved in enzymatic reactions;

FIG. 2 is a schematic diagram of a biomolecular control system under a Brink controller;

FIG. 3 shows a regulation diagram of an idealized enzymatic reaction process under Brink control based on DNA strand displacement;

FIG. 4 is a graph of a long-term regulation result of experiments relating to the regulation of an idealized enzymatic reaction process under Brink control based on DNA strand displacement;

FIG. 5 shows a regulation diagram of an idealized enzymatic reaction process under Brink control based on DNA strand displacement enzymatic reaction process under Brink control based on DNA strand displacement;

FIG. 6 is a graph of the process regulation result of an enzymatic reaction under Brink control with an initial concentration of substance D₁ being 1.1 nM;

FIG. 7 is a graph of the process regulation result of an enzymatic reaction under Brink control with an initial concentration of substance D₁ being set as 1.1 nM and an initial concentration of substance F being set as 1.3 nM.

### DETAILED DESCRIPTION OF THE EMBODIMENTS

In order that the objects, aspects, and advantages of the present application will become more apparent, the present application will be further described in detail with reference to the accompanying drawings and embodiments. It should be understood that the specific embodiments described here are only used to explain the present application and are not intended to limit the present application, that is, the described embodiments are only a part of the embodiments of the present application, not all of them.

Thus, the following detailed description of the embodiments of the present application provided in the accompanying drawings is not intended to limit the scope of the claimed application but is merely representative of selected embodiments of the application. Based on the embodiments of this application, all other embodiments obtained by those skilled in the art without manufacturing any inventive effort fall within the scope of protection of the present application.

### EXAMPLE 1

This example provides an implementation method for ultrasensitive Brink control for a delayed enzymatic reaction based on DNA strand displacement, specifically including:

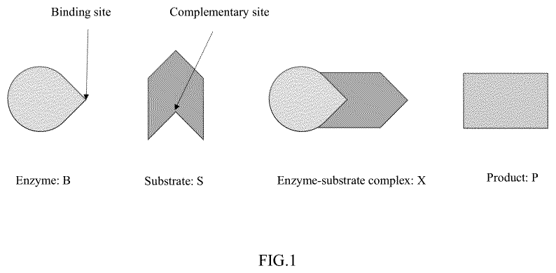

S1: describing an enzymatic reaction process using single-molecule and bimolecular chemical reactions and obtaining:

$$\begin{matrix}S + B {⇄}_{{K}_{2}}^{{K}_{1}} X \\ X {\to }^{{k}_{3}} P + B \\ P {\to }^{{k}_{4}} ∅\end{matrix}$$

where S and B represent a substrate and an enzyme, respectively, X and P represent an enzyme-substrate complex and a product, respectively, as shown in FIG. 1.

Considering that the enzymatic reaction process is easily affected by factors such as temperature and pH value, the enzymatic activity in the above reaction will be changed, thus affecting the production efficiency of the product in the enzymatic reaction. Further, the accumulation of substrate S takes some time (S is influenced by the input flux U according to reaction

$$U {\to }^{1.} S ) .$$

In order to more accurately simulate the reaction process, a delay factor τ can be introduced into the reaction model, and an enzymatic reaction process model with a time delay can be constructed:

$$\begin{matrix}S + B {\to }^{{k}_{1}} X \\ X {\to }^{{k}_{2}} S + B \\ X {\to }^{{k}_{3}} P + B \\ P {\to }^{{k}_{4} , τ} ∅\end{matrix}$$

where parameter τ represents a cumulative time delay present in the production of the output substance P.

S2: constructing a CRN-based Brink controller.

In particular, the Brink controller is designed to directly reduce the number of CRNs needed to implement a hypersensitive I/O response compared to a controller based on a dual-rail representation, such as a QSM controller, since it does not involve the use of a subtraction module, reducing the complexity of the DNA implementation.

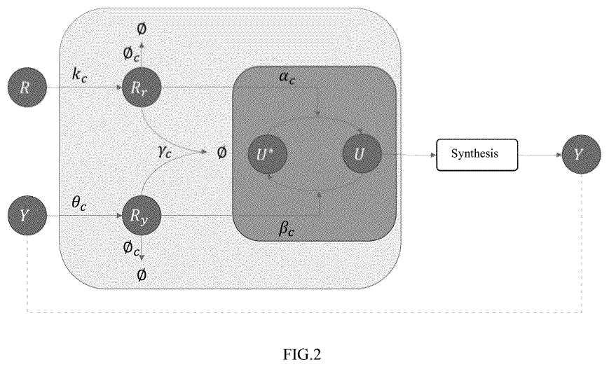

The biggest feature of the Brink controller in the design of CRNs is to circumvent the limitation of the dual-rail representation method. As shown in FIG. 2, parameters R and Y is an input to the Brink controller and U represents an output. The parameters k_{c}, θ_{c}, and α_{c} represent catalysis rates; γ_{c} and β_{c} represent binding rate; ϕ_{c} represents a degradation rate. Further, R can produce Rᵣ, which in turn is reacted with U* to form U; while Y can produce R_{y}, which in turn is reacted with U to form U*. At the same time, substances Rᵣ and R_{y} are bound form a complex Rᵣ·R_{y}, which does not interact with any other substance, i.e. there is an inverse functional mechanism between the two different inputs R and Y of the Brink controller. The modules highlighted in FIG. 2 belong to a covalent modification cycle, and the Brink controller uses signals Rᵣ and R_{y} as activator and deactivator, respectively. The corresponding CRNs can be represented as follows:

$$\begin{matrix}R {\to }^{{k}_{c}} R + {R}_{r} \\ Y {\to }^{{θ}_{c}} Y + {R}_{y} \\ {R}_{r} + {R}_{y} {\to }^{{γ}_{c}} {R}_{r} \cdot {R}_{y} \\ {R}_{r} {\to }^{{ϕ}_{c}} ∅ \\ {R}_{y} {\to }^{{ϕ}_{c}} ∅ \\ {R}_{r} + {U}^{*} {\to }^{{α}_{c}} U + {R}_{r} \cdot {R}_{y} \\ {R}_{y} + U {\to }^{{β}_{c}} {U}^{*}\end{matrix}$$

Combined with the mass action kinetics (MAKs), the corresponding ODEs equations are:

$$\begin{matrix}\frac{{d [ {R}_{r} ]}_{t}}{d t} = {{k}_{c} [ R ]}_{t} - {{{γ}_{c} [ {R}_{r} ]}_{t} [ {R}_{y} ]}_{t} - {{ϕ}_{c} [ {R}_{r} ]}_{t} - {{{α}_{c} [ {R}_{r} ]}_{t} [ {U}^{*} ]}_{t} \\ \frac{{d [ {R}_{y} ]}_{t}}{d t} = {{θ}_{c} [ Y ]}_{t} - {{{γ}_{c} [ {R}_{r} ]}_{t} [ {R}_{y} ]}_{t} - {{ϕ}_{c} [ {R}_{y} ]}_{t} - {{{β}_{c} [ {R}_{y} ]}_{t} [ U ]}_{t} \\ \frac{{d [ {U}^{*} ]}_{t}}{d t} = - {{{α}_{c} [ {R}_{r} ]}_{t} [ {U}^{*} ]}_{t} + {{{β}_{c} [ {R}_{y} ]}_{t} [ U ]}_{t} \\ \frac{{d [ U ]}_{t}}{d t} = {{{α}_{c} [ {R}_{r} ]}_{t} [ {U}^{*} ]}_{t} - {{{β}_{c} [ {R}_{y} ]}_{t} [ U ]}_{t}\end{matrix}$$

(d[U*]ₜ/dt)+(d[U]ₜ/dt)=0 obtained from the differential equation indicates that the total mass U+U* is conserved during the process of time evolution.

S3: obtaining a static mapping expression between an output of the Brink controller and an output of the system under a steady state condition so as to obtain an analytical condition ensuring the performance of the controller.

Specifically, assuming that the Brink controller has achieved a steady state output, the following results can be obtained:

k_{c}[R]ₜ−γ_{c}[Rᵣ]ₜ[R_{y}]ₜ−ϕ_{c}[Rᵣ]ₜ−α_{c}[Rᵣ]ₜ[U*]ₜ=0

θ_{c}[Y]ₜ−γ_{c}[Rᵣ]ₜ[R_{y}]ₜ−ϕ_{c}[R_{y}]ₜ−β_{c}[R_{y}]ₜ[U]ₜ=0

−α_{c}[Rᵣ]ₜ[U*]ₜ+β_{c}[R_{y}]ₜ[U]ₜ=0

Assuming that the reference input R to the Brink controller is constant, the following equation constraint can be obtained:

$$\begin{matrix}[ \overline{Y} ] = \frac{{k}_{c} [ R ] - {α}_{c} [ {\overline{R}}_{r} ] [ {\overline{U}}^{*} ] + {β}_{c} [ {\overline{R}}_{y} ] [ \overline{U} ] - {ϕ}_{c} [ {\overline{R}}_{r} ] + {ϕ}_{c} [ {\overline{R}}_{y} ]}{{θ}_{c}} \\ = \frac{{k}_{c}}{{θ}_{c}} [ R ] + \frac{{ϕ}_{c}}{{θ}_{c}} ( [ {\overline{R}}_{y} ] - [ {\overline{R}}_{r} ] )\end{matrix}$$

where the signal [⋅̅] is indicative of the concentration of the substance ⋅ at a steady state.

Compared with QSM controllers that can also achieve ultrasensitive response, the steady-state equilibrium conditions of Brink controllers are only related to two variables Rᵣ and R_{y}. There are relatively fewer factors that affect the final output results. The reduction of the number of variables contained in the constraints of the steady-state regulation equation helps to maintain the desired steady-state output, thus simplifying the control structure and reducing the complexity of the controller design.

S4: constructing a Brink controller by DNA strand displacement reaction.

Specifically, i, x, y, and z are set as variables, where i∈(1, 2, . . . , 12), x∈(1, 2, . . . , 8), y∈(1, 2, 3, 4), and z∈(1, 2, . . . , 9); For the following DNA implementation, Gₓ, Tₓ and L_{y} represent auxiliary substances involved in the reaction; O_{z} and H_{y} represent intermediate products; B_{y} represents inert wastes produced by the reaction which do not interact with other substances; further, Cₘₐₓ represents the initial concentration of the auxiliary substance; qₘₐₓ represents the reaction rate of maximum strand displacement; qᵢ represents the reaction rate of the corresponding DNA implementation.

For reactions

$$R {\to }^{{k}_{c}} R + {R}_{r} and Y {\to }^{{θ}_{c}} Y + {R}_{y} ,$$

there is a common DSD implementation mechanism between the two. These two reactions can be converted into:

$$\begin{matrix}\begin{matrix}R + {G}_{1} {\to }^{{q}_{1}} ∅ + {O}_{1} \\ {O}_{1} + {T}_{1} {\to }^{{q}_{max}} R + {R}_{r}\end{matrix} \} , {q}_{1} = \frac{{k}_{c}}{{C}_{max}} \\ \begin{matrix}Y + {G}_{2} {\to }^{{q}_{2}} ∅ + {O}_{2} \\ {O}_{2} + {T}_{2} {\to }^{{q}_{max}} Y + {R}_{y}\end{matrix} \} , {q}_{2} = \frac{{θ}_{c}}{{C}_{max}}\end{matrix}$$

At the same time, there is also an identical realization mechanism between the reactions

$${R}_{r} {\to }^{{ϕ}_{c}} ∅ and {R}_{y} {\to }^{{ϕ}_{c}} ∅ ,$$

and the transformation can be represented as:

$$\begin{matrix}{R}_{r} + {G}_{3} {\to }^{{q}_{3}} ∅ \\ {R}_{y} + {G}_{4} {\to }^{{q}_{4}} ∅\end{matrix} \} , \qquad {q}_{3} = {q}_{4} = \frac{{ϕ}_{c}}{{C}_{max}}$$

For reactions

$${R}_{r} + {R}_{y} {\to }^{{γ}_{c}} {R}_{r} \cdot {R}_{y} , {R}_{r} + {U}^{*} {\to }^{{α}_{c}} U + {R}_{r} \cdot {R}_{y} and {R}_{y} + U {\to }^{{β}_{c}} {U}^{*} ,$$

the corresponding DNA implementations are represented as:

$$\begin{matrix}{R}_{r} + {L}_{1} {⇄}_{{q}_{max}}^{{q}_{5}} {H}_{1} + {B}_{1} \\ {R}_{y} + {H}_{1} {⟶}^{{q}_{max}} {O}_{3} + ∅ \\ {O}_{3} + {T}_{3} {⟶}^{{q}_{max}} {R}_{r} \cdot {R}_{y} + ∅\end{matrix} \} , {q}_{5} = {γ}_{c}$$

$$\begin{matrix}{R}_{r} + {L}_{2} {⇄}_{{q}_{max}}^{{q}_{6}} {H}_{2} + {B}_{2} \\ {U}^{*} + {H}_{2} {⟶}^{{q}_{max}} {O}_{4} + ∅ \\ {O}_{4} + {T}_{4} {⟶}^{{q}_{max}} {R}_{r} \cdot {R}_{y} + U\end{matrix} \} , {q}_{6} = {α}_{c}$$

$$\begin{matrix}{R}_{y} + {L}_{3} {⇄}_{{q}_{max}}^{{q}_{7}} {H}_{3} + {B}_{3} \\ U + {H}_{2} {⟶}^{{q}_{max}} {O}_{5} + ∅ \\ {O}_{5} + {T}_{5} {⟶}^{{q}_{max}} {U}^{*} + ∅\end{matrix} \} , {q}_{7} = {β}_{c}$$

S5: obtaining a time delay representation through a DNA strand displacement mechanism based on a delayed substance and a compensation mechanism, and applying the time delay representation to a DNA implementation of the enzymatic reaction process model; at the same time, combined with the constructed Brink controller, controlling a delayed enzymatic reaction process model.

Specifically, in order to use the DNA strand displacement reaction to represent the time delay, two circuits consisting of two abstract chemical reactions taking place simultaneously are designed to represent the time delay. The implementation of this mechanism is based on delaying the participation of substances. The following reaction can be described by:

$${∅}_{source} {\to }^{{k}_{prod}} O$$

$$O + D {\to }^{{k}_{delay}} {∅}_{waste}$$

where parameters k_{prod} and k_{delay} are rate constants. In a first stage, substance O is produced at a constant rate; in the second stage, when O is bound to delay substance D, it is rapidly converted to waste Ø_{waste}. This mechanism takes the time the substance D is consumed by the substance O as the delay time, the delay effect of which depends on the initial concentration of delay substance D.

The delayed enzymatic reaction model can be rewritten as:

$$S + B {\to }^{{k}_{1}} X$$

$$X {\to }^{{k}_{2}} S + B$$

$$X {\to }^{{k}_{3}} P + B$$

$$P + {D}_{1} {\to }^{{k}_{delay 1}} ∅$$

$$P {\to }^{{k}_{4}} ∅$$

where k_{delay1} represents a delayed reaction rate. Combined with the mass action kinetics (MAKs), the following results can be obtained:

$$\frac{{d [ S ]}_{t}}{dt} = - {{{k}_{1} [ S ]}_{t} [ B ]}_{t} + {{k}_{2} [ X ]}_{t} + {[ U ]}_{t}$$

$$\frac{{d [ B ]}_{t}}{dt} = - {{{k}_{1} [ S ]}_{t} [ B ]}_{t} + {{k}_{2} [ X ]}_{t} + {{k}_{3} [ X ]}_{t}$$

$$\frac{{d [ X ]}_{t}}{dt} = {{{k}_{1} [ S ]}_{t} [ B ]}_{t} - {{k}_{2} [ X ]}_{t} - {{k}_{3} [ X ]}_{t}$$

$$\frac{{d [ P ]}_{t}}{dt} = {{k}_{3} [ X ]}_{t} - {{{k}_{delay 1} [ P ]}_{t} [ {D}_{1} ]}_{t} - {{k}_{4} [ P ]}_{t}$$

$$\frac{{d [ {D}_{1} ]}_{t}}{dt} = - {{{k}_{delay 1} [ P ]}_{t} [ {D}_{1} ]}_{t}$$

By using the reaction DNA strand displacement reaction mechanism, the reaction

$$X {\to }^{{k}_{3}} P + B can$$

be converted to:

$$\begin{matrix}X + {G}_{5} {\to }^{{q}_{8}} ∅ + {O}_{6} \\ {O}_{6} + {T}_{6} {\to }^{{q}_{max}} P + B\end{matrix} \} , {q}_{8} = \frac{{k}_{3}}{{C}_{max}}$$

Degradation reaction

$$P {\to }^{{k}_{4}} ∅$$

can be converted to:

$$P + {G}_{6} {\to }^{{q}_{9}} ∅ , {q}_{9} = \frac{{k}_{4}}{{C}_{max}}$$

Further, reaction

$$P + {D}_{1} {\to }^{{k}_{delay 1}} ∅$$

can be converted to:

$$\begin{matrix}P + {G}_{7} {\to }^{{q}_{10}} ∅ + {O}_{7} \\ {O}_{7} + {D}_{1} {\to }^{{q}_{max}} ∅\end{matrix} \} , {q}_{10} = \frac{{k}_{delay 1}}{{C}_{max}}$$

For reversible reaction

$$S + B {⇄}_{{K}_{2}}^{{K}_{1}} X ,$$

the original reaction form is maintained when designing the DNA implementation.

It should be noted that for all reaction rates involved in the CRN-based Brink controller and enzymatic process, the substrate values are shown in Table 1 and Table 2, and the feasible values are Cₘₐₓ=1000 nM, and qₘₐₓ=10⁷/M/s, respectively. For the Brink controller, the initial value of signals Rᵣ, R_{y}, and Rᵣ·R_{y} are set to zero, i.e. Rᵣ₀=R_{y0}=[Rᵣ·R_{y}]₀=0 nM.

TABLE 1
Parameter representation of the enzymatic reaction process model

| Parameters | Description | Nominal value |
|---|---|---|
| k₁ | Binding rate | 5.0 × 10⁻³ s⁻¹ |
| k₂ | Unbinding rate | 1.0 × 10⁻⁴ s⁻¹ |
| k₃ | Catalytic reaction rate | 1.6 s⁻¹ |
| k₄ | Degradation reaction rate | 2.0 × 10⁻⁵ s⁻¹ |
| B_{Total} | Sum of the amount of B + X | 3.0 nM |

Further, the initial concentration of the sum of substances X and P in the enzymatic reaction is set to zero, i.e. X₀=P₀=0 nM. Then the related experiments were designed and the results were analyzed.

TABLE 2
Parametric representation of Brink controller

| Parameters | Description | Nominal value |
|---|---|---|
| k_{c} | Catalytic reaction rate | 5.0 × 10⁻³ s⁻¹ |
| θ_{c} | Catalytic reaction rate | 5.0 × 10⁻³ s⁻¹ |
| γ_{c} | Binding rate | 2.0 × 10⁴ M⁻¹s⁻¹ |
| ϕ_{c} | Degradation reaction rate | 3.0 × 10⁻⁴ s⁻¹ |
| α_{c} | Catalytic reaction rate | 1.2 × 10⁴ M⁻¹s⁻¹ |
| β_{c} | Binding rate | 1.2 × 10⁴ M⁻¹s⁻¹ |
| U_{Total} | Sum of the total amount of | 5.0 nM |
| | U + U* | |

1) No delay

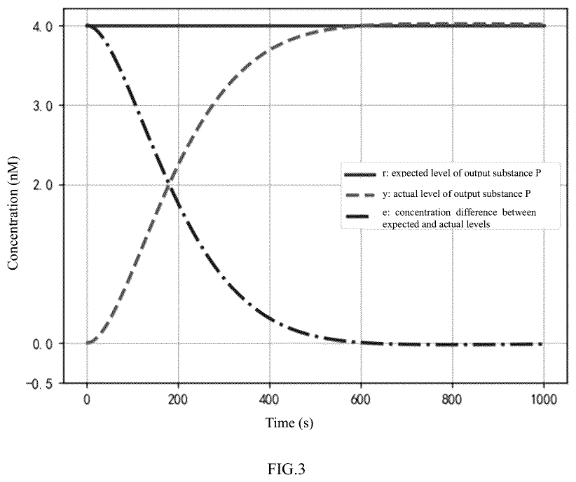

For the course of the enzymatic reaction a fixed constant can be chosen for the expected concentration of the output substance P, i.e. the reference signal R can be set to 4.0 nM. At this point an ideal enzymatic reaction model is analyzed, i.e. the initial concentration of D₁ is zero. The corresponding results are shown in Table 3. In FIG. 3, the signal Y is outputted, that is, the substance P has an actual concentration that gradually approaches the ideal output concentration and maintains a stable output state over time.

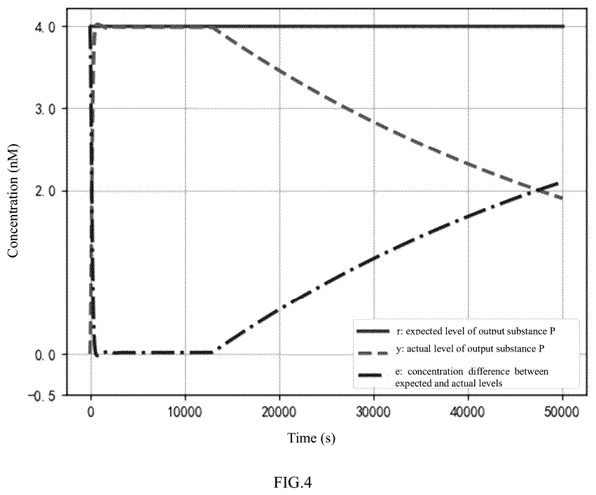

It is to be noted that the adjustment results as shown in FIG. 3 only indicate that the desired output state can be reached in a limited time. In fact, if the simulation is run long enough, the entire regulation will collapse, resulting in a transition of the ideal output state to another, as shown in FIG. 4. The adjustment of the whole system can at least ensure that the actual concentration Y of the output substance P is close to the expected concentration within a certain time. The phenomenon observed in FIG. 4, mainly due to the consumption of the fuel strand, leads to a gradual decrease in the total concentration of fuel over time, which in turn leads to a gradual decrease in the performance of the DNA circuit.

2) Non-Zero Delay

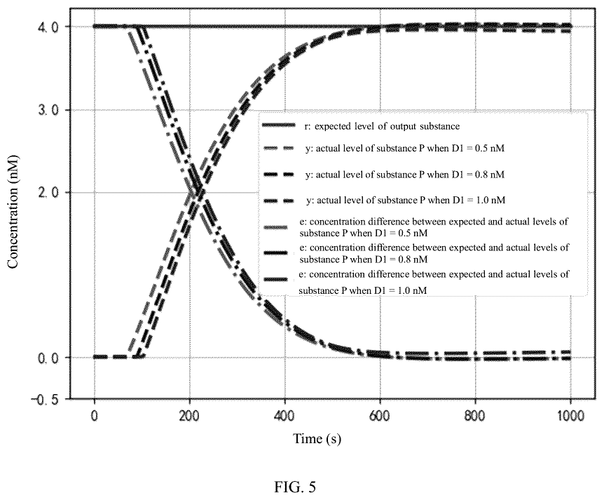

Next, the course of the enzymatic reaction with a non-zero delay was analyzed, i.e. the initial concentration of the substance D₁ is not zero. The delayed reaction rate k_{delay1} is set to 1.0×10² s⁻¹. The initial concentration of the delay material D₁ is set to three different values, 0.5 nM, 0.8 nM, and 1.0 nM, and the system responses under the corresponding conditions are shown in FIG. 5. According to the parameters shown in Table 3, the delayed effects of the system responses become more pronounced as the concentration of the substance increases. [D₁]₀ represents the initial concentration of substance D₁.

TABLE 3
Parameterization of non-zero delay model adjustment results

| [D₁]₀ (nM) | Delay output time (s) |
|---|---|
| 0.5 | 70 |
| 0.8 | 92 |
| 1.0 | 105 |

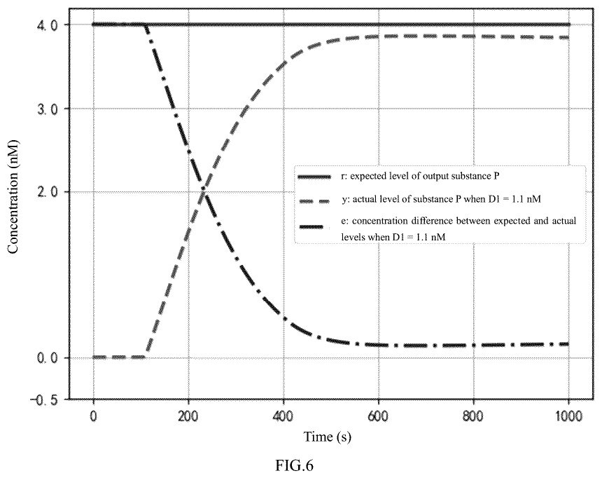

However, the delay mechanism described above is achieved by consuming a delay substance, and this process requires the participation and consumption of output substance P to some extent. For DNA-based enzymatic reactions with a time delay, this allows the actual output of the output substance P to be lower than expected. The effect shown in FIG. 5 may not be apparent, but when D₁=1.1 nM the output response of the whole system is as shown in FIG. 6.

In order to solve this problem, the following reaction mechanism is designed to compensate for the consumption of the substance P in the reaction

$$P + {D}_{1} {\to }^{{k}_{delay 1}} ∅ ,$$

thereby achieving the desired yield of the output substance P.

$$P + F {\to }^{{k}_{pro 1}} 2 P$$

$$P {\to }^{{k}_{pro 2}} F$$

where kₚᵣₒ₁ and kₚᵣₒ₂ are both reaction rate constants and F is an additionally added reaction substance. Combined with the mass action kinetics (MAKs), the corresponding ordinary differential equations (ODEs) can be obtained:

$$\frac{{d [ P ]}_{t}}{dt} = {{{k}_{pro 1} [ P ]}_{t} [ F ]}_{t} - {{k}_{pro 2} [ P ]}_{t}$$

$$\frac{{d [ F ]}_{t}}{dt} = - {{{k}_{pro 1} [ P ]}_{t} [ F ]}_{t} + {{k}_{pro 2} [ P ]}_{t}$$

Further, the reaction

$$P + F {\to }^{{k}_{pro 1}} 2 P$$

can be converted to:

$$\begin{matrix}P + {L}_{4} {⇄}_{{q}_{max}}^{{q}_{11}} {H}_{4} + {B}_{4} \\ F + {H}_{4} {\to }^{{q}_{max}} {O}_{8} + ∅ \\ {O}_{8} + {T}_{7} {\to }^{{q}_{max}} 2 P + ∅\end{matrix} \} , {q}_{11} = {k}_{pro 1}$$

Reaction

$$P {\to }^{{k}_{pro 2}} F$$

can be converted to:

$$\begin{matrix}P + {G}_{8} {⟶}^{{q}_{12}} ∅ + {O}_{9} \\ {O}_{9} + {T}_{8} {⟶}^{{q}_{max}} F + ∅\end{matrix} \} , {q}_{12} = \frac{{k}_{pro 2}}{{C}_{max}}$$

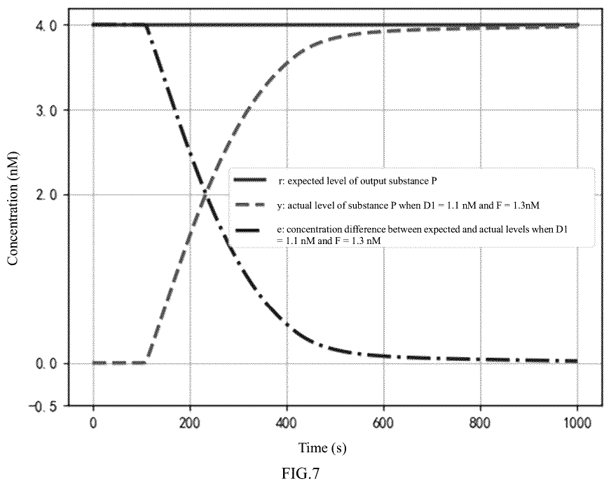

For the redesigned control scheme described above, the corresponding adjustment results are shown in FIG. 7. The reaction rate kₚᵣₒ₁ is set to 6.2×10⁻⁵ s⁻¹, and the reaction rate kₚᵣₒ₂ is set to 3.0×10⁻⁵ s⁻¹.

The curve for the actual yield of the output substance P in FIG. 7 is significantly different from the results in FIG. 6. Over time another curve can gradually approach the expected concentration of the substance P. This difference is due to the construction of the compensation mechanism, the addition of chemical substance F. Therefore, it is feasible to express the DNA strand displacement with respect to time delay based on the delayed substance and compensation mechanism, and at the same time, the enzymatic reaction process can achieve the expected output result under the action of the Brink controller.

The Brink controller in the present invention circumvents the limitation of the dual-rail representation method, greatly reducing the number of required CRNs, DNA reactions and DNA strands, and reducing the complexity of DNA implementation.

The foregoing descriptions of specific exemplary embodiments of the present invention have been presented for purposes of illustration and example. It is not intended to limit the invention to the precise form disclosed, and obviously, many modifications and variations are possible in light of the above teaching. The purpose of selecting and describing exemplary embodiments is to explain the specific principles and practical applications of the present invention so that those skilled in the art can implement and utilize various exemplary embodiments of the present invention, as well as various choices and changes. The scope of the present invention is intended to be limited by the claims and the equivalents.

What is claimed is:

1. An implementation method for ultrasensitive Brink control for a delayed enzymatic reaction based on DNA strand displacement, comprising:
describing an enzymatic reaction process using single-molecule and bimolecular chemical reactions, and introducing a time delay factor to obtain an enzymatic reaction process model with time delay;
constructing a CRN-based Brink controller;
obtaining a static mapping expression between an output of the Brink controller and an output of the system under a steady state condition so as to obtain an analytical condition ensuring the performance of the controller;
constructing a Brink controller by DNA strand displacement reaction; and
obtaining a time delay representation through a DNA strand displacement mechanism based on a delayed substance and a compensation mechanism, and applying the time delay representation to a DNA implementation of the enzymatic reaction process model; at the same time, combined with the constructed Brink controller, controlling a delayed enzymatic reaction process model.

2. The implementation method for ultrasensitive Brink control for a delayed enzymatic reaction based on DNA strand displacement of claim 1, wherein describing the enzymatic reaction process using single-molecule and bimolecular chemical reactions specifically comprises:
$$S + B {{⇄}_{{K}_{2}}}^{{K}_{1}} X$$
$$X {⟶}^{{k}_{3}} P + B$$
$$P {⟶}^{{k}_{4}} ∅$$
where S and B represent a substrate and an enzyme, respectively, X and P represent an enzyme-substrate complex and an output substance, respectively.

3. The implementation method for ultrasensitive Brink control for a delayed enzymatic reaction based on DNA strand displacement of claim 1, wherein constructing the enzymatic reaction process model with time delay specifically comprises:
$$S + B {⟶}^{{k}_{1}} X$$
$$X {⟶}^{{k}_{2}} S + B$$
$$X {⟶}^{{k}_{3}} P + B$$
$$P {⟶}^{{k}_{4} , τ} ∅$$
where the parameter τ represents a cumulative time delay present in the production of the output substance P.

4. The implementation method for ultrasensitive Brink control for a delayed enzymatic reaction based on DNA strand displacement of claim 1, wherein the CRN-based controller is represented as:
$$R {⟶}^{{k}_{c}} R + {R}_{r}$$
$$Y {⟶}^{{θ}_{c}} Y + {R}_{y}$$
$${R}_{r} + {R}_{y} {⟶}^{{γ}_{c}} {R}_{r} \cdot {R}_{y}$$
$${R}_{r} {⟶}^{{ϕ}_{c}} ∅$$
$${R}_{y} {⟶}^{{ϕ}_{c}} ∅$$
$${R}_{r} + {U}^{*} {⟶}^{{a}_{c}} U + {R}_{r} \cdot {R}_{y}$$
$${R}_{y} + U {⟶}^{{β}_{c}} {U}^{*}$$
where parameters R and Y are inputs to the Brink controller and U represents an output; parameters k_{c}, θ_{c} and α_{c} represent the catalysis rate; γ_{c} and β_{c} represents the binding rate; ϕ_{c} represents the degradation rate; further, the parameter R produces substance Rᵣ, which in turn is reacted with U* to form U; parameter Y produces substance R_{y}, which in turn is reacted with U to form U*; at the same time, signals Rᵣ and R_{y} are bound to form a complex Rᵣ·R_{y} that does not interact with any other substance, i.e. there is an inverse functional mechanism between the two different input parameters R and Y of the Brink controller; the Brink controller uses the signals Rᵣ and R_{y} as activator and deactivator, respectively;
combined with the mass action kinetics (MAKs), the corresponding ODEs equations are:
$$\frac{{d [ {R}_{r} ]}_{t}}{dt} = {{k}_{c} [ R ]}_{t} - {{{γ}_{c} [ {R}_{r} ]}_{t} [ {R}_{y} ]}_{t} - {{ϕ}_{c} [ {R}_{r} ]}_{t} - {{{a}_{c} [ {R}_{r} ]}_{t} [ {U}^{*} ]}_{t}$$
$$\frac{{d [ {R}_{y} ]}_{t}}{dt} = {{θ}_{c} [ Y ]}_{t} - {{{γ}_{c} [ {R}_{r} ]}_{t} [ {R}_{y} ]}_{t} - {{ϕ}_{c} [ {R}_{y} ]}_{t} - {{{β}_{c} [ {R}_{y} ]}_{t} [ U ]}_{t}$$
$$\frac{{d [ {U}^{*} ]}_{t}}{dt} = - {{{a}_{c} [ {R}_{r} ]}_{t} [ {U}^{*} ]}_{t} + {{{β}_{c} [ {R}_{y} ]}_{t} [ U ]}_{t}$$
$$\frac{{d [ U ]}_{t}}{dt} = {{{a}_{c} [ {R}_{r} ]}_{t} [ {U}^{*} ]}_{t} - {{{β}_{c} [ {R}_{y} ]}_{t} [ U ]}_{t}$$
(d[U*]ₜ/dt)+(d[U]ₜ/dt)=0 obtained from the differential equation indicates that the total mass U+U* is conserved during the process of time evolution.

5. The implementation method for ultrasensitive Brink control for a delayed enzymatic reaction based on DNA strand displacement of claim 1, wherein obtaining the static mapping expression between the output of the Brink controller and the output of the system under the steady state, specifically comprises:
assuming the Brink controller has achieved steady-state output, the following results are obtained:
k_{c}[R]ₜ−γ_{c}[Rᵣ]ₜ[R_{y}]ₜ−ϕ_{c}[Rᵣ]ₜ−α_{c}[Rᵣ]ₜ[U*]ₜ=0
θ_{c}[Y]ₜ−γ_{c}[Rᵣ]ₜ[R_{y}]ₜ−ϕ_{c}[R_{y}]ₜ−β_{c}[R_{y}]ₜ[U]ₜ=0
−α_{c}[Rᵣ]ₜ[U*]ₜ+β_{c}[R_{y}]ₜ[U]ₜ=0
assuming that the Brink controller reference input R is constant, the following constraints are obtained:
$$[ \overline{Y} ] = \frac{{k}_{c} [ R ] - {a}_{c} [ {\overline{R}}_{r} ] [ {\overline{U}}^{*} ] + {β}_{c} [ {\overline{R}}_{y} ] [ \overline{U} ] - {ϕ}_{c} [ {\overline{R}}_{r} ] + {ϕ}_{c} [ {\overline{R}}_{y} ]}{{θ}_{c}} = \frac{{k}_{c}}{{θ}_{c}} [ R ] + \frac{{ϕ}_{c}}{{θ}_{c}} ( [ {\overline{R}}_{y} ] - [ {\overline{R}}_{r} ] )$$
where the signal [⋅̅] is indicative of the concentration of the substance ⋅ at a steady state.

6. The implementation method for ultrasensitive Brink control for a delayed enzymatic reaction based on DNA strand displacement of claim 1, wherein constructing the Brink controller by DNA strand displacement reaction, specifically comprises: set i, x, y, z as variables, where i∈(1, 2, . . . , 12), x∈(1, 2, . . . , 8), y∈(1, 2, 3, 4), z∈(1, 2, . . . , 9);
for reactions
$$R {⟶}^{{k}_{c}} R + {R}_{r} and Y {⟶}^{{θ}_{c}} Y + {R}_{y} ,$$
there is a common DSD implementation mechanism between the two; these two reactions are converted into:
$$\begin{matrix}R + {G}_{1} {⟶}^{{q}_{1}} ∅ + {O}_{1} \\ {O}_{1} + {T}_{1} {⟶}^{{q}_{max}} R + {R}_{r}\end{matrix} \} , {q}_{1} = \frac{{k}_{c}}{{C}_{max}}$$
$$\begin{matrix}Y + {G}_{2} {⟶}^{{q}_{2}} ∅ + {O}_{2} \\ {O}_{2} + {T}_{2} {⟶}^{{q}_{max}} Y + {R}_{y}\end{matrix} \} , {q}_{1} = \frac{{θ}_{c}}{{C}_{max}}$$
at the same time, there is also an identical realization mechanism between the reactions
$${R}_{r} {⟶}^{{ϕ}_{c}} ∅ and {R}_{y} {⟶}^{{ϕ}_{c}} ∅ ,$$
and the transformation is represented as:
$$\begin{matrix}{R}_{r} + {G}_{3} {⟶}^{{q}_{3}} ∅ \\ {R}_{y} + {G}_{4} {⟶}^{{q}_{4}} ∅\end{matrix} \} , {q}_{3} = {q}_{4} = \frac{{ϕ}_{c}}{{C}_{max}}$$
for the reactions
$${R}_{r} + {R}_{y} {⟶}^{{γ}_{c}} {R}_{r} \cdot {R}_{y} , {R}_{r} + {U}^{*} {⟶}^{{a}_{c}} U + {R}_{r} \cdot {R}_{y} and {R}_{y} + U {⟶}^{{β}_{c}} {U}^{*} ,$$
the corresponding DNA implementations are represented as:
$$\begin{matrix}{R}_{r} + {L}_{1} {{⇄}_{{q}_{max}}}^{{q}_{5}} {H}_{1} + {B}_{1} \\ {R}_{y} + {H}_{1} {⟶}^{{q}_{max}} {O}_{3} + ∅ \\ {O}_{3} + {T}_{3} {⟶}^{{q}_{max}} {R}_{r} \cdot {R}_{y} + ∅\end{matrix} \} , {q}_{5} = {γ}_{c}$$
$$\begin{matrix}{R}_{r} + {L}_{2} {{⇄}_{{q}_{max}}}^{{q}_{6}} {H}_{2} + {B}_{2} \\ {U}^{*} + {H}_{2} {⟶}^{{q}_{max}} {O}_{4} + ∅ \\ {O}_{4} + {T}_{4} {⟶}^{{q}_{max}} {R}_{r} \cdot {R}_{y} + U\end{matrix} \} , {q}_{6} = {a}_{c}$$
$$\begin{matrix}{R}_{y} + {L}_{3} {{⇄}_{{q}_{max}}}^{{q}_{7}} {H}_{3} + {B}_{3} \\ U + {H}_{2} {⟶}^{{q}_{max}} {O}_{5} + ∅ \\ {O}_{5} + {T}_{5} {⟶}^{{q}_{max}} {U}^{*} + ∅\end{matrix} \} , {q}_{7} = {β}_{c}$$
where Gₓ, Tₓ and L_{y} represent auxiliary substances involved in the reaction; O_{z} and H_{y} represent intermediate products; B_{y} represents inert wastes produced by the reaction which do not interact with other substances; further, Cₘₐₓ represents the initial concentration of the auxiliary substance; qₘₐₓ represents the reaction rate of maximum strand displacement; qᵢ represents the reaction rate of the corresponding DNA implementation.

7. The implementation method for ultrasensitive Brink control for a delayed enzymatic reaction based on DNA strand displacement of claim 1, wherein obtaining the time delay representation through the DNA strand displacement mechanism and based on the delayed substance and a compensation mechanism, specifically comprises:
the time delay is represented by a circuit consisting of two abstract chemical reactions taking place simultaneously, the implementation of which is based on the participation of a delaying substance D, and described by the following reactions:
$${∅}_{source} {⟶}^{{k}_{prod}} O$$
$$O + D {⟶}^{{k}_{delay}} {∅}_{waste}$$
where parameters k_{prod} and k_{delay} are rate constants; in a first stage, substance O is produced at a constant rate; in the second stage, when the substance O is bound to the delayed substance D, it is rapidly converted into waste Ø_{waste}; the time taken for the substance O to consume substance D is taken as the delay time, and the delay effect thereof depends on the initial concentration of the delay substance D.

8. The implementation method for ultrasensitive Brink control for a delayed enzymatic reaction based on DNA strand displacement of claim 1, wherein by applying the time delay representation to the DNA implementation of the enzymatic reaction process model, the enzymatic reaction model is rewritten as:
$$S + B {⟶}^{{k}_{1}} X$$
$$X {⟶}^{{k}_{2}} S + B$$
$$X {⟶}^{{k}_{3}} P + B$$
$$P + {D}_{1} {⟶}^{{k}_{delay 1}} ∅$$
$$P {⟶}^{{k}_{4}} ∅$$
where k_{delay1} represents a delayed reaction rate; combined with the mass action kinetics (MAKs), the following results are obtained:
$$\frac{{d [ S ]}_{t}}{dt} = - {{{k}_{1} [ S ]}_{t} [ B ]}_{t} + {{k}_{2} [ X ]}_{t} + {[ U ]}_{t} \\ \frac{{d [ B ]}_{t}}{dt} = - {{{k}_{1} [ S ]}_{t} [ B ]}_{t} + {{k}_{2} [ X ]}_{t} + {{k}_{3} [ X ]}_{t} \\ \frac{{d [ X ]}_{t}}{dt} = {{{k}_{1} [ S ]}_{t} [ B ]}_{t} - {{k}_{2} [ X ]}_{t} - {{k}_{3} [ X ]}_{t} \\ \frac{{d [ P ]}_{t}}{dt} = {{k}_{3} [ X ]}_{t} - {{{k}_{delay 1} [ P ]}_{t} [ {D}_{1} ]}_{t} - {{k}_{4} [ P ]}_{t} \\ \frac{{d [ {D}_{1} ]}_{t}}{dt} = - {{{k}_{delay 1} [ P ]}_{t} [ {D}_{1} ]}_{t} .$$

9. The implementation method for ultrasensitive Brink control for a delayed enzymatic reaction based on DNA strand displacement of claim 1, wherein combined with the constructed Brink controller, controlling the delayed enzymatic reaction process model, specifically comprises:
converting reaction
$$X {⟶}^{{k}_{3}} P + B$$
to:
$$\begin{matrix}X + {G}_{5} {⟶}^{{q}_{8}} ∅ + {O}_{6} \\ {O}_{6} + {T}_{6} {⟶}^{{q}_{max}} P + B\end{matrix} \} , {q}_{8} = \frac{{k}_{3}}{{C}_{max}}$$
converting degradation reaction
$$P {⟶}^{{k}_{4}} ∅$$
to:
$$P + {G}_{6} {⟶}^{{q}_{9}} ∅ , {q}_{9} = \frac{{k}_{4}}{{C}_{max}}$$
further, converting reaction
$$P + {D}_{1} {⟶}^{{k}_{delay 1}} ∅$$
to:
$$\begin{matrix}P + {G}_{7} {⟶}^{{q}_{10}} ∅ + {O}_{7} \\ {O}_{7} + {D}_{1} {⟶}^{{q}_{max}} ∅\end{matrix} \} , {q}_{10} = \frac{{k}_{delay 1}}{{C}_{max}}$$
for reversible reaction
$$S + B {\begin{matrix}⟶ \\ ⟵\end{matrix}}_{{K}_{2}}^{{K}_{1}} X ,$$
the original reaction corm is maintained when designing the DNA implementation.

10. The implementation method for ultrasensitive Brink control for a delayed enzymatic reaction based on DNA strand displacement of claim 1, wherein the enzymatic reaction process model of the Brink-based controller is adjusted by using DSD mechanism; the proposed expression of DNA strand displacement with respect to time delay is improved, specifically as follows: the consumption of substance P in stage
$$P + {D}_{1} {⟶}^{{k}_{delay 1}} ∅$$
in the enzymatic reaction is compensated by the following reaction mechanism to achieve the desired yield of output substance P:
$$P + F {⟶}^{{k}_{pro 1}} 2 P \\ P {⟶}^{{k}_{pro 2}} F$$
where kₚᵣₒ₁ and kₚᵣₒ₂ are both reaction rate constants and F is an additionally added reaction substance; combined with the mass action kinetics (MAKs), the corresponding ordinary differential equations (ODEs) are obtained:
$$\frac{{d [ P ]}_{t}}{dt} = {{{k}_{pro 1} [ P ]}_{t} [ F ]}_{t} - {{k}_{pro 2} [ P ]}_{t} \\ \frac{{d [ F ]}_{t}}{dt} = - {{{k}_{pro 1} [ P ]}_{t} [ F ]}_{t} + {{k}_{pro 2} [ P ]}_{t}$$
further, reaction is
$$P + F {⟶}^{{k}_{pro 1}} 2 P$$
is converted to:
$$\begin{matrix}P + {L}_{4} {\begin{matrix}⟶ \\ ⟵\end{matrix}}_{{q}_{max}}^{{q}_{11}} {H}_{4} + {B}_{4} \\ F + {H}_{4} {⟶}^{{q}_{max}} {O}_{8} + ∅ \\ {O}_{8} + {T}_{7} {⟶}^{{q}_{max}} 2 P + ∅\end{matrix} \} , {q}_{11} = {k}_{pro 1}$$
reaction
$$P {⟶}^{{k}_{pro 2}} F$$
is converted to:
$$\begin{matrix}P + {G}_{8} {⟶}^{{q}_{12}} ∅ + {O}_{9} \\ {O}_{9} + {T}_{8} {⟶}^{{q}_{max}} F + ∅\end{matrix} \} , {q}_{12} = \frac{{k}_{pro 2}}{{C}_{max}} .$$
